(19) United States
(12) Patent Application Publication
(10) Pub. No.: US 2005/0148887 A1
(43) Pub. Date: Jul. 7, 2005

(54) Detection and alarm system

(75) Inventors: Harald Reiter, Aachen (DE); Gereon Vogtmeier, Aachen (DE); Josef Lauter, Geilenkirchen (DE); Olaf Such, Aachen (DE); Christan Reichinger, Neutraubling (DE); Ralf Schmidt, Aachen (DE)

Correspondence Address: PHILIPS INTELLECTUAL PROPERTY & STANDARDS, P.O. BOX 3001, BRIARCLIFF MANOR, NY, 10510, US

(73) Assignee: Koninklijke Philips Electronics N.V., BA Eindhoven (NL)

(21) Appl. No.: 10/509,232

(22) PCT Filed: Mar. 6, 2003
(86) PCT No.: PCT/IB03/00842

(30) Foreign Application Priority Data
Mar. 29, 2002 (EP) 02076227.4

(51) Int. Cl. A61B 5/02

(52) U.S. Cl. 600/508

(57) ABSTRACT

A system for continuous monitoring of a physiological condition of a recipient arranged to alarm the recipient and a remote service center in case an abnormality in the physiological condition occurs. For that purpose the system comprises monitoring means provided with a set of electrodes to be arranged on the body of the recipient and to derive a signal related to said condition, detection means actuated by said electrodes and arranged to process said signal in order to derive a feature in the signal characteristic to said abnormality; alarm means arranged to trigger an alarm signal upon a detection of said feature by the detection means; transmission means arranged to transmit the alarm signal to a station responsive to said alarm signal. The system architecture is arranged in such a way that only the alarm signal is transferred to the station thus allowing the monitoring in a continuous way.

The invention relates to a system for detecting an abnormality in a physiological condition of a user and for alerting people to said abnormality, said system comprising monitor means for monitoring a signal representative of the physiological condition, said monitor means comprising sensor means arranged to be located on the body of the user for detecting said signal; detection means actuated by said sensor means and arranged to process said signal in order to derive a feature in the signal characteristic to said abnormality; alarm means arranged to trigger an alarm signal upon a detection of said feature by the detection means; transmission means arranged to transmit the alarm signal to a station responsive to said alarm signal.

A system of a kind described in the opening paragraph is known from U.S. Pat. No. 5,228,449. The known system is arranged to monitor the physiological condition of the user and for alerting people in case a problem in said condition occurs. The known system comprises monitoring means provided with a set of electrodes. The monitoring means are arranged on the body of the user, a so-called user-side of the system in order to pick up a signal related to the physiological condition, for example ECG-signals. The known system further comprises transmitting means also arranged on the user-side of the system to transmit the picked-up signal to a base unit located outside the user-side of the system. The base-unit performs an analysis of the transmitted signal in order to derive a feature characteristic to a problem. For example, for cardiac applications a certain cardiac rate is a feature characteristic to a condition of a cardiac arrest. In case the analysis indicates that there is a problem in the physiological condition of the user an alarm is generated, which can be sound locally, for example at the user's home, or at a remote location.

The known system has a disadvantage that for continuous monitoring of a physiological condition a continuous data transmission has to take place resulting in a high power consumption of the system leading to a necessity to frequently replace the local power supply of the system.

It is an object of the invention to provide a system as described in the opening paragraph, where the power consumption of the system is reduced leading to an improved durability of the system.

The system according to the invention is characterized in that the detection means are arranged on a user-side of the system, the alarm signal being the sole signal transmitted by the monitor means to said station. According to the technical measure of the invention a system architecture is designed to achieve an ultra-low power consumption leading to a feasibility of a true continuous monitoring of a physiological condition. An example of said condition is a cardiac activity, body temperature, respiration rate, encephalogram, etc. The system according to the invention is arranged to perform a 24-hour monitoring, where the signal analysis is performed on a body-worn unit without a need for a permanent wires-less connection to a unit performing a data analysis. The data analysis is performed by means of a per se known software. The software can include a pre-set table where characteristic features are identified. Therefore, according to the technical measure of the invention the power-consuming transmission to a station outside the user-side of the system occurs only in a situation where an abnormality in the physiological condition of the user is detected. It is also possible that the features are ranked up according to the severity of the abnormality of the physiological condition being monitored. For example, for cardiac applications, a minor change in the cardiac cycle can be recognized as an alarm of the lowest category, where an occurrence of arrythmia or fibrillation can be ranked higher. The alarm signal can be ranked accordingly to the rank of the feature.

An embodiment of the system according to the invention is characterized in that the station is a stationary home-based station arranged to forward the alarm signal automatically to a remote service center. In situations when the user is suffering from a problem in the physiological condition being monitored, the home-base station receives an alarm signal to warn the user and/or a member of a family or a neighbor (to ask for assistance. In situations where the problem is the problem of a high severity, for example a cardiac arrest a prompt and adequate medical response is necessary. According to an embodiment of the invention, the home-based station is arranged to forward the alarm signal to the remote service center. An example of the remote service center is a call center arranged to manage medical emergencies of a kind. The remote service center will take over the management of the emergency and can inform the respective communal or medical sites about the emergency, the address of the user, patient data and the probable diagnose. This enables the fast and adequate handling of life-threatening abnormalities and adds to an improved survival chance during emergencies.

A further embodiment of the system according to the invention is characterized in that said home-based station is further arranged to control a domestic device. An example of a domestic device is an electronically adjustable door lock, or a tap water control or a control of other domestic appliances, like irons, ovens, etc. According to this technical feature the user is ensured that the medical personnel can enter his home to give the medical assistance and that the potentially dangerous domestic apparatus are switched off so that no danger to the user's environment can be caused. It is also possible that in case the monitoring system is supplied together with an Automatic External Defibrillator (AED) the station actuates a telephone module of the AED in order to instruct a family member of the user how to operate the device in case the user is suffering from an cardiac arrest.

A still further embodiment of the invention is characterized in that the station is a mobile station arranged to forward the alarm signal to a remote service center and in that said system further comprises positioning means actuated by the alarm means, said positioning means being arranged to determine a location of the user and to transmit a signal representative to said location to said remote service center. According to this technical feature the user is ensured of an adequate handling of the problem also in case he is outside his home. In this case after a characteristic feature to a certain abnormality has been derived from the acquired signal and the corresponding alarm signal has been transmitted to the mobile station provided together with the system, the mobile station forwards the alarm together with an information about the location of the user to the remote service center. The information about the location of the user is collected by the positioning means arranged in the system. An example of the positioning means is an interface to a Global Positioning System (GPS) which can provide the co-ordinates of the user. These co-ordinates will then be transmitted together with the alarm signal to the remote service center. The service center will then forward the alarm signal together with the co-ordinates of the user location to the medical sites responsible for a proper handling of emergencies. Alternatively, for locations inside buildings where no GPS signal can be acquired the positioning means can be arranged to link up to a stationary in-door locating system. An example of such a locating system is known from U.S. Pat. No. 6,292,687. The known system is arranged to comprise a network of position localizers, for example installed in every hotel room, each position localizer emitting a characteristic beacon signal identifying the location. In case the positioning means are linked-up to such an in-door locating system the position of the user in-door can be registered accurately and transmitted to a service station together with the alarm signal in case of an emergency.

A still further embodiment of the system according to the invention is characterized in that the user-side of the system further comprises range detection means arranged to validate that the user is located within an operational range of the station. This technical measure has an advantage that the user-side of the system is enabled to check whether the user is still located within the operational range of the station. This is of particular importance in case a mobile station is not attached to the user-side of the system. Due to this technical measure it is prevented that the user leaves home without the mobile station on him.

A still further embodiment of the system according to the invention is characterized in that the monitor means further comprise a motion sensor arranged to monitor a physical activity of the user. This technical feature has the advantage that in case the monitoring system detects a cardiac arrest this condition is double-checked by means of a motion detector. Also, the motion detector can be arranged to prevent the monitoring system from gathering false data in case of a too extensive body movement.

A still further embodiment of the system according to the invention is characterized in that the monitoring means are integrated in a wearable garment. By integrating the sensors in a clothing, for example an elastic belt of an underwear slip or a brassier a patient-friendly monitoring system can be obtained for continuous monitoring purposes. By means of the elastic belt the sensors are constantly put under the necessary pressure to ensure a constant position of the sensors with respect to the user's skin. In case the wiring is integrated in the fabric of the elastic belt as well, a monitoring system can be obtained providing a maximum convenience and privacy to the user. An example of a suitable electrode material is a per se known electrically conductive rubber which has a certain degree of stretchability as well, adding to the patient's comfort. By sealing off the electrical contacts between the electrode material and the wiring a washable wearable monitoring system can be provided.

These and other aspects of the invention will be discussed with reference to the attached figures.

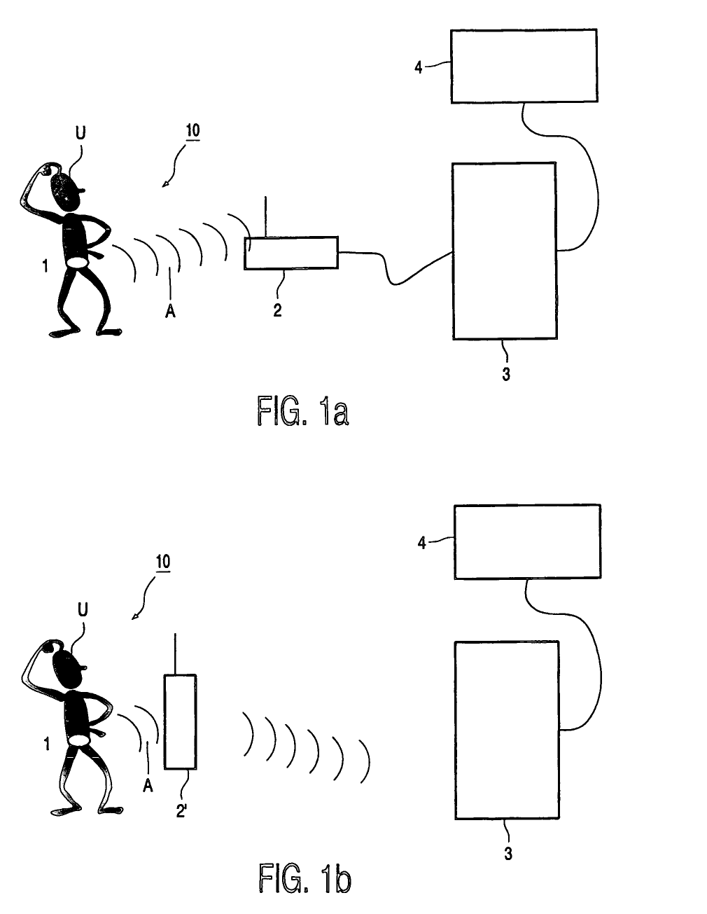

FIG. 1a shows a schematic view of an embodiment of the system architecture in case the user is located at his home.

FIG. 1b shows a schematic view of an embodiment of the system architecture in case the user in located outside.

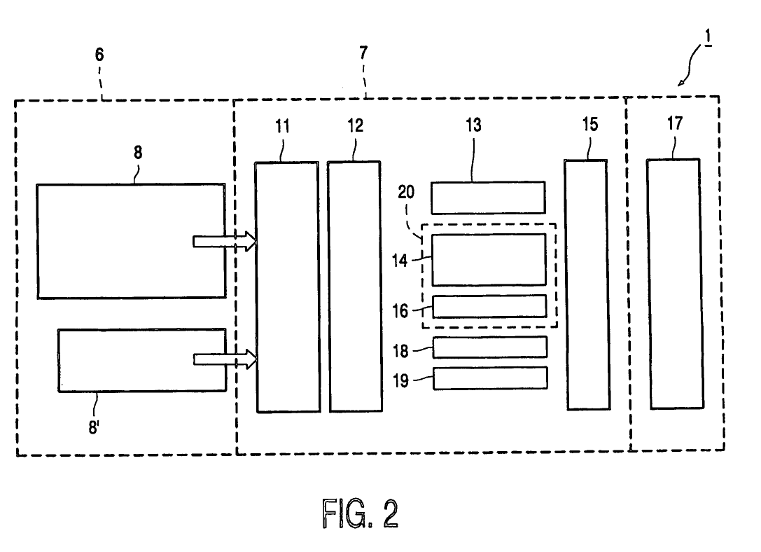

FIG. 2 shows schematically an embodiment of the components of the user-side of the system according to the invention.

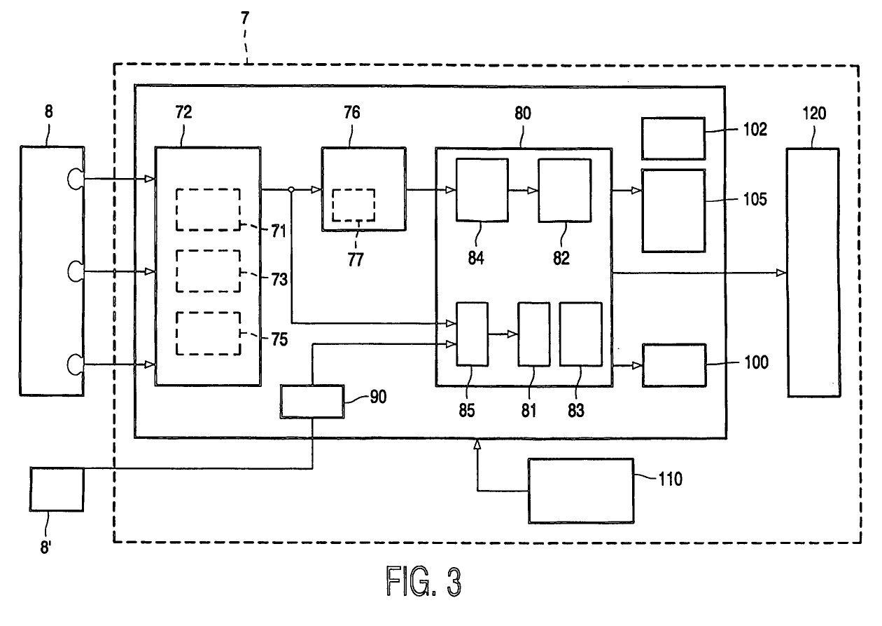

FIG. 3 shows schematically an embodiment of the electronics of the front-end of the system of FIG. 2 in more detail.

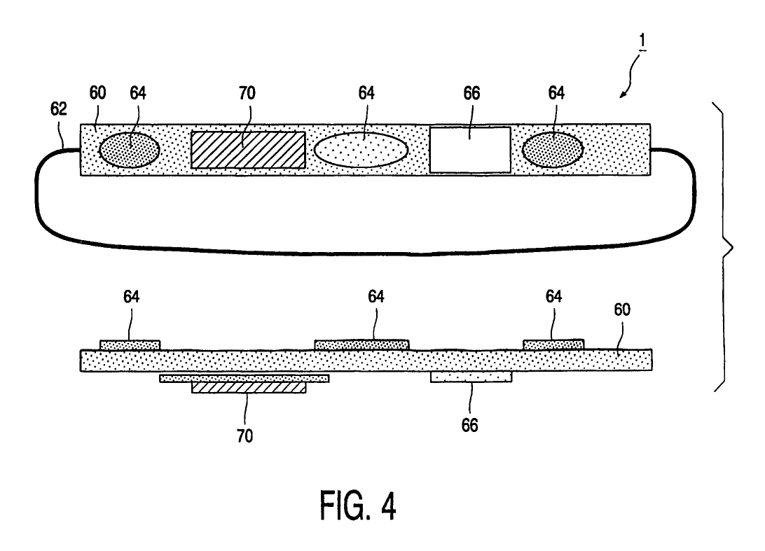

FIG. 4 shows schematically an embodiment of the user-side of the system integrated into a wearable garment.

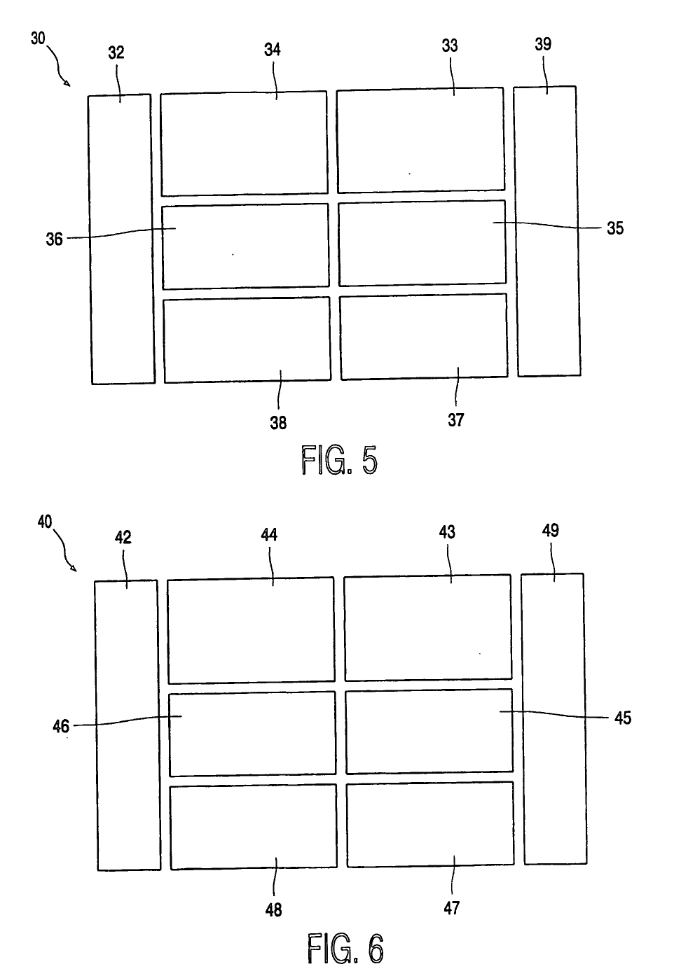

FIG. 5 shows schematically an embodiment of the components of the home station module.

FIG. 6 shows schematically an embodiment of the components of the mobile station module.

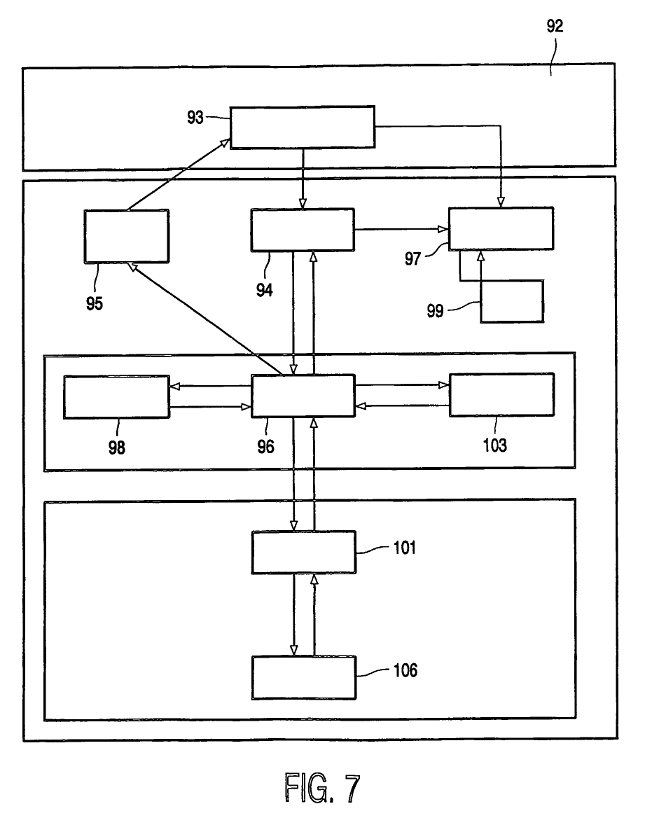

FIG. 7 shows schematically an embodiment of an operational flow-chart of the system according to the invention.

FIG. 1a shows a schematic view of an embodiment of the system architecture in case the user is located at his home. The user U is provided with a detection and alarm system 10 comprising a user-side 1 and a non-user side 2. The user-side 1 comprises monitoring means and a front-end electronics, both described in more detail with reference to FIGS. 2 and 3. The user-side 1 of the system is arranged to transmit an alarm signal A, preferably via a RF-link to the non-user side 2 of the system 10. The non-user side 2 comprises a home station 2 arranged to pick-up the alarm signal A and to forward the alarm signal to a remote service center 3 arranged to handle the medical emergency, for example by forwarding the alarm signal to a public emergency center 4.

FIG. 1b shows a schematic view of an embodiment of the system architecture in case the user is located outdoors. The user U is provided with a detection and alarm system 10 comprising a user-side 1 and a non-user side 2′. The user-side 1 comprises monitoring means and a front-end electronics, both described in more detail with reference to FIGS. 2 and 3. The user-side 1 of the system 10 is arranged to transmit an alarm signal A, preferably via a RF-link to the non-user side 2′ of the system. The non-user side 2′ comprises a mobile station 2′ arranged to pick-up the alarm signal A and to forward the alarm signal to a remote service center 3 together with an information about a location of the user U by means of a wireless connection. An example of a suitable transmitting technology is GSM for the mobile station and the plain old telephone service (POTS) for the home station, etc. The remote service center 3 is arranged to handle the medical emergency, for example by forwarding the alarm signal to a public emergency center 4.

FIG. 2 shows schematically an embodiment of the components of the user-side 1 of the system according to the invention. The user-side 1 comprises monitoring means 6 arranged to monitor a physiological condition of the user. The monitoring means 6 comprise a set of electrodes 8 arranged on the body of the user to pick-up a signal characteristic of the physiological signal, for example an ECG signal, a body temperature, respiration rate, encephalogram, etc. Additionally, the monitor means 6 can comprise a sensor 8′ arranged to monitor a signal not directly related with a targeted physiological condition. An example of such a sensor is a motion sensor, or a blood pressure sensor. The monitoring means 6 are arranged to perform a continuous monitoring of a physiological condition of the user and are further arranged to provide a corresponding signal to the front-end electronics 7 of the user-side 1 of the system. The monitoring means 6 and the front-end electronics 7 are worn on the body of the user, preferably at the waist area. An example of the preferred embodiment of a body worn system is shown in FIG. 6. The front-end electronics 7 is arranged to analyze said signal in order to derive a feature characteristic to an abnormality in the physiological condition of the user. For that purpose the front-end electronics 7 comprise a preamplifier and analogue processing circuit 11, an ADC unit 12, a L-processor 13, detection means 20, alarm means 15 and transmission means 17. The detection means 20 comprise a sensor signal interpretation unit 14 and feature extraction means 16. The user-side 1 of the system operates as follows: the monitoring means 6 acquire the raw data which are delivered to the front-end 7. The front-end provides means for receiving the signals from the monitoring means, performs suited analogue processing by means of the analogue processing circuit 11. The processed raw data is converted into a digital format by means of the ADC 12 and is forwarded by a μ-processor 13 to the detection means 20, where the condition of the user is being analyzed. For example, for cardiac applications the detection means 20 can comprise a per-se known QRS-detector to determine R-R peak intervals in heart cycles. The detection means 20 comprise a sensor signal interpretation unit 14 arranged to derive a feature in the signal characteristic to an abnormal physiological condition of the user. For example, for cardiac or cranial applications said feature can be a frequency of the signal, for heamodynamic studies said feature can be a threshold value of a blood pressure and so on. It is also possible that more than one feature is assigned per monitored physiological condition. In this case the features can be ranked up according to the severity of the abnormality of the physiological condition being monitored. For example, for cardiac applications, a minor change in the cardiac cycle can be recognized as an alarm of the lowest category, whereas an occurrence of arrythmia or fibrillation can be ranked higher. The alarm signal can be ranked accordingly to the rank of the feature. In both situations, the value of the feature or the features can be stored in a look-up table (not shown) of the memory unit 18. Additionally, the system can be arranged as a self-learning system, where the threshold value for the feature is being adjusted and stored in the look-up table in cases a pre-stored value does not correspond to an abnormal condition for a particular user.

In case the detection means 20 detects the abnormal condition, a signal is sent to the alarm means 15 to generate an alarm, which is transmitted by the transmitting means 17, for example by means of a RF-link. The alarm signal is transmitted to the home station in case the user experiences an abnormality at home, or, alternatively to a mobile station for locations of the user away from home. From the respective station the emergency center is informed and is provided with the exact position of the customer (at home/actual position outside home). The alarm center takes over the management of the emergency and informs the respective communal or medical sites about the emergency, the location, patient data and the probable diagnose. This enables the fastest possible treatment (early defibrillation) and gives an improved chance to safe customers' lives.

FIG. 3 shows schematically an embodiment of the electronics of the front-end of the system in more detail. The front-end electronics 7 is composed of an analogue input stage 72 which comprises circuits for a lead off detection 71 to check the electrical contact with the electrodes 8, an amplifier 73 and a low pass filter 75. The low pass filter 75 is preferably arranged with a 3 dB cut off frequency of 30 Hz. The signals at the output of the unit 72 are fed via a multiplexer unit 85 to an ADC unit 81 of the μ-processor 80. The μ-processor 80 comprises a software implemented to allow a sophisticated signal analysis, for example a digital signal processing. For cardiac applications an example of the digital signal processing is a digital QRS detector and a digital detector for ventricular fibrillation. Additionally, the front-end 7 comprises a band pass filter 76, preferably with a center frequency of 16 Hz and a 3 dB bandwidth of 18 Hz. The signals from the band pass filter 76 are fed to an analogue signal detector 77. For cardiac applications the signal detector 77 comprises a QRS detector that is composed of a full wave rectifier and a peak detector (both not shown in the figure). The pulses from the signal detector 77 are fed to the input 84 of the μ-processor 80 where the signals are further analyzed to derive a feature characteristic of the physiological condition of the user. For cardiac applications such a feature can be an interval between subsequent characteristic peaks in the ECG spectrum, for example an interval between subsequent R-peaks. The results of the signal analysis can be stored in a memory block 105.

Additionally an interface 90 to a motion sensor 8′ can be integrated into the front-end electronics 7. An example of a suitable sensor is a ADXL202 from Analog Devices. Alternatively, the motion sensor can be positioned directly at the electronics, a skin contact is not necessary. In that case the unit 90 is the motion sensor in FIG. 3. An on/off switch 102 detects whether the user is wearing the electrodes 8 and performs a shut down operation if the system is not used, for example while the user-side of the system is being replaced. The power management unit 110 is arranged to supply a stable voltage supply and comprises standard low power devices.

The operation of the front-end electronics will be explained using an example of the cardiac monitoring. The electrodes 8 supply a one-lead ECG signal to the front-end 7 in a continuous mode. To allow ultra power consumption the ECG signals are only analyzed by the analogue QRS detector 77. As long as the QRS pulses are within a defined limit, stored in an internal memory block 82 of the μ-processor 80, the system assumes that the condition of the user is normal and no further action is applied. This is referred to as an operational mode IDLE with a minimum power consumption. In case the QRS pulses fall outside the pre-stored limit or in case the signal quality degrades, the motion sensor interface 90 is actuated by the μ-processor 80. The interface 90 detects whether there is a movement and the signal degradation or an (increased) heart rate is due to movement artifacts or due to an extensive exercise. In case the signal quality degrades substantially the lead-off detector 71 is actuated to check whether the electrodes are still placed and connected to the user's body. In case the motion detector gives no positive or unique result, and the lead-off detector 71 validates that the electrodes are on the user's body, the ECG signal is sampled, preferably with 100 samples per second by the ADC unit 81 of the μ-processor 80 and is supplied to the digital detector 83 for special digital processing. This operation is referred to as an ALERT mode. In case the processing detects an emergency according to the derived feature an alarm signal is sent to the home or mobile station via a RF-link 120. This mode of operation is referred to as an EMERGENCY mode. The above operational modes of the front-end will be discussed in more detail with reference to FIG. 8. The respective station is arranged to provide a local alarm and to forward the alarm signal to a remote service center responsible for an adequate medical response to an emergency.

FIG. 4 shows schematically an embodiment of the user-side 1 (see FIG. 4a) of the system integrated into a wearable garment, preferably into an elastic belt of an underwear slip or a brassier. The components of the user-side according to the present embodiment of the system are attached to the elastic belt 60 that can be locked with a help of an elastic band 62. A set of electrodes 64 is provided to monitor a signal characteristic to a targeted physiological condition of the user. For cardiac applications, for example the set of electrodes 64 can be arranged to measure an ECG signal. In this case it is sufficient to use three electrodes that obtain a one-lead ECG measurement. The electrodes can be of a dry type and can be glued or attached differently on the belt 60. Wires (not shown) can be provided in different ways, for example the wiring can be integrated into the fabric of the elastic belt or an external wiring can be used. Wires establish electrical connections to the front-end electronics 70 as well as provide electrical connections from a battery 66 to the front-end electronics 70 for the power supply. The front-end module 70 can comprise the units discussed with reference to FIG. 3. In order to satisfy to the requirements of a wearing comfort, the battery 66 can be manufactured as a flexible battery, for example a lithium ion battery and the front-end electronics 70 can be carried by a flexible foil (shown in FIG. 4b). It is also possible to divide the front-end electronics 70 into a number of units, preferably two units, each unit separately carried by the flexible foil for a better weight distribution of the units across the belt 60.

FIG. 5 shows schematically an embodiment of the components of the home station module 30. The home station module 30 is arranged to work in a continuous mode 24-hours a day and operates in a full-automatic mode without user interaction. The position of the home station is known, which is the user's address. Therefore, a localization of the user in case of an abnormality is not necessary. The home station 30 is active if the customer stays at home and is actuated by means of an RF-link 32 which is controllable by the user-side of the system (not shown in the figure). The RF link 32 is the counterpart of the front-end RF link and can be an off-the-shelf 868 MHz RF module using e.g. FM modulation to connect to the front-end. The operation for the home station 30 is controlled by a μ-processor 36. The home station 30 comprises means 34 to generate a local alarm in case the abnormality is detected. Further, the home station 30 comprises means 33 to forward the alarm signal to a remote service center (not shown) together with an information about the user's address provided by the unit 35. The unit 39 is arranged to actuate a telephone link to the remote service center for further handling of the alarm. The link 39 to the alarm center can be a standard telephone modem. There is a possibility for the user to overrule the generated alarm by means of the user interface 37, which is necessary in case a false alarm is generated. The home station 30 further comprises a range detection means 38 arranged to verify whether the user is within the range of the RF-link 32.

It is also possible to use the range detection unit 38 to warn the user that he is outside the range. The RF link of the front-end of the user-side of the system (not shown in the figure) can be arranged to send a short message with an identifier and to listen afterwards for a reply from the RF-link 32 of the home station 30. In case the reply has been received the front-end will return to a stand-by mode, otherwise a special beeper on the front-end will generate a warning. The ranging function can be started at fixed time intervals, for example every minute, or with a variable time intervals, for every 5 minutes or can be controlled by the motion detector allowing for longer periods during a rest of the user. According to this technical measure a sophisticated and reliable monitoring and alarm system can be produced. The operational configuration of the home station 30 is controlled by a μ-controller (not shown) which is responsible for configuration of alarm messages, controlling the modem and the RF transceiver, self-testing and out-of-range detection, when applicable. Local alarm functionality can be provided by acoustic and visible alarming means (e.g. beeper, loud speaker) in a range up to 50 m to inform nearby people about the cardiac emergency situation. Indicator e.g. LEDs can be provided to show the actual status of the system. Optionally, a LCD display can be provided to show status messages and display vital parameters such as heart rate.

FIG. 6 shows schematically an embodiment of the components of the mobile station module. The mobile station 40 is actuated in case the user stays outside his home and has the same functionality as the home station. The mobile station comprises an RF-link 42, a local alarm means 44, a μ-processor 46, emergency call configuration means 43, user interface 47, wireless link 49 to a remote service center. The mobile station can also be equipped with an out of range detection means 48 which operate according to the same principle as a similar unit of the home station of FIG. 5. Due to the fact that the user is located outside his home, his exact dwell position must be known in case an emergency occurs. Therefore the mobile station 40 comprises positioning means 45 arranged to determine the actual position of the user on-line. An example of the positioning means is an interface to a Global Positioning System (GPS) which can provide the co-ordinates of the user. These co-ordinates will then be transmitted together with the alarm signal to the remote service center. The service center will then forward the alarm signal together with the co-ordinates of the user location to the medical sites responsible for a proper handling of emergencies. Alternatively, for locations inside buildings where no GPS signal can be acquired the positioning means can be arranged to link up to a stationary in-door locating system. The mobile station 40 can be implemented to comprise two parts: a standard handy (guaranteed communication coverage) with an integrated modem and a localization part (e.g. a GPS receiver and micro controller for determination of the actual position). The localization part can be integrated in a handy clip or a cellular phone pocket. The mobile station operates in a full-automatic mode without user interaction if the user stays outside home. The position of the mobile station is not known, therefore the localization of the customer has to be determined on a regular base this can be performed by means of the GPS system.

FIG. 7 shows schematically an embodiment of an operational flow-chart of the system according to the invention. To minimize the power consumption the system operates in different modes. After a boot operation, schematically presented by a block 92 the system performs a self-test procedure 94. Examples of check procedures are a power check, electrode on/off check, links check, periphery check, etc. In case the check procedure is passed the system switches to the IDLE mode 96, otherwise the system enters an error state 97. In the latter case a warning message can be sent by means of an audible or a visual signal by means as shown by step 99. The IDLE mode 96 is a standard mode of the system characterized by a minimum power consumption. Preferably, only in the IDLE mode user interactions 103 and service interactions 98 can be executed. The system remains in the IDLE mode in case the signal from the monitoring means shows no abnormalities. In case the user decides to switch off the system, this action is detected at a switch-off step 95, leading the system to a shut-down mode (not shown). In case an abnormality is detected during the monitoring, the system changes to an ALERT mode 101, where an extended monitoring takes place. In case an abnormality is not caused by a deterioration of the physiological condition of the user, the system return to the IDLE mode 96. Otherwise, the system enters an EMERGENCY mode 104 where an alarm signal is generated and communicated to external devices, such as the home station or the mobile station. Therefore, the system architecture is developed to ensure that the system has a low power consumption during a normal operational state leading to an increased durability of the system. This feature is of a particular importance for ensuring reliable monitoring in a continuous mode during extended periods of time.

1. A system for detecting an abnormality in a physiological condition of a user and for alerting people to said abnormality, said system comprising monitor means for monitoring a signal representative of the physiological condition, said monitor means comprising:
sensor means arranged to be located on the body of the user for detecting said signal;
detection means actuated by said sensor means and arranged to process said signal in order to derive a feature in the signal characteristic to said abnormality;
alarm means arranged to trigger an alarm signal upon a detection of said feature by the detection means; and
transmission means arranged to transmit the alarm signal to a station responsive to said alarm signal, characterized in that the detection means are arranged on a user-side of the system, the alarm signal being the sole signal transmitted by the monitor means to said station.

2. A system according to claim 1, characterized in that the station is a stationary home-based station arranged to forward the alarm signal to a remote service center.

3. A system according to claim 2, characterized in that said home-based station is further arranged to control a domestic device.

4. A system according to claim 1, characterized in that the station is a mobile station arranged to forward the alarm signal to a remote service center and in that said system further comprises positioning means actuated by the alarm means, said positioning means being arranged to determine a location of the user and to transmit a signal representative to said location to said remote service center.

5. A system according to claim 1, characterized in that the user-side of the system further comprises range detection means arranged to validate that the user is located within an operational range of the station.

6. A system according to claim 1, characterized in that said abnormality in the physiological condition is a condition of the cardiac arrest.

7. A system according to claim 5, characterized in that the system further comprises an automatic external defibrillator device provided with a telephone module actuatable by the remote service center in case of an emergency.

8. A system according to claim 1 characterized in that the monitor means further comprise a motion sensor arranged to monitor a physical activity of the user.

9. A system according to claim 8, characterized in that the monitoring means are integrated in a wearable garment.
